(19) United States
(12) Patent Application Publication
(10) Pub. No.: US 2025/0367469 A1
(43) Pub. Date: Dec. 4, 2025

(54) VIAL ASSEMBLY SYSTEMS AND METHODS FOR OPTIMAL FLOW

(71) Applicant: BARD PERIPHERAL VASCULAR, INC., Franklin Lakes, NJ (US)

(72) Inventors: Casey HEBERT, Loveland, CO (US); Brandon SIMMONS, Tempe, AZ (US); Amanda THYSTRUP, Phoenix, AZ (US); Mark Nicholas WRIGHT, Coventry, RI (US)

(73) Assignee: BARD PERIPHERAL VASCULAR, INC., Franklin Lakes, NJ (US)

(21) Appl. No.: 18/875,551

(22) PCT Filed: Jul. 22, 2022
(86) PCT No.: PCT/US2022/038019

§ 371 Date: Dec. 16, 2024

(51) Int. Cl. A61N 5/10; A61J 1/14; A61J 1/20

(52) U.S. Cl. CPC A61N 5/1002; A61J 1/1406; A61J 1/201; A61J 1/2037; A61J 1/2079; A61J 1/2093; A61N 2005/1021

(57) ABSTRACT

Systems and methods involve a vial assembly system comprising a vial assembly and needle. The vial assembly comprises a septum, neck region, and particulate region comprising a bypass valve, the neck region comprising a top, bottom, and sidewall. The needle comprises a port and a tip configured to puncture the septum that is configured to receive the needle such that the port is disposed in the neck region. A bypass path includes a first end and a second end, the bypass valve is connected to the first end, the second end ends at or within the sidewall, the bypass path is configured to provide a fluid flow path to the needle alternate to a first path upon a build-up of pressure for fluid flow in the vial assembly, and the first path is directly disposed in the vial assembly between the particulate region and the neck region.

### TECHNICAL FIELD

The present disclosure generally relates to components of medical devices for treating cancer, and more particularly to vial assembly systems of medical devices configured and operable to deliver radioactive compounds to a treatment area within a patient's body in procedures such as transarterial radioembolization.

### BACKGROUND

In cancer treatments involving radiation therapy, inadvertent or excess exposure to radiation from radioactive therapeutic agents can be harmful and potentially lethal to patients or medical personnel. Accordingly, medical instruments for radiation therapies must be configured to localize the delivery of radioactive material to a particular area of the patient's body while shielding others from unnecessarily being exposed to radiation.

Transarterial Radioembolization is a transcatheter intra-arterial procedure performed by interventional radiology and is commonly employed for the treatment of malignant tumors. During this medical procedure, a microcatheter is navigated into a patient's liver where radioembolizing microspheres loaded with a radioactive compound, such as yttrium-90 (⁹⁰Y), are delivered to the targeted tumors. The microspheres embolize blood vessels that supply the tumors while also delivering radiation to kill tumor cells. Generally, a clinician or patient may be at risk from radiation emitted from the delivery.

Accordingly, a need exists for components of a medical device configured and operable to shield from such radiation and optimize flow when delivering the radioactive compound to the patient's body.

### SUMMARY

In accordance with an embodiment of the disclosure, a vial assembly system comprises a vial assembly and a needle. The vial assembly comprises a septum, a neck region, and a particulate region for containing particulates comprising a bypass valve, the neck region comprising a top, a bottom, and a sidewall disposed therebetween. The needle comprises a port and a tip distal of the port, the tip configured to puncture the septum of the vial assembly disposed at the bottom of the neck region. The septum is configured to receive the needle such that the port is disposed in the neck region of the vial assembly. A bypass path includes a first end and a second end. The bypass valve is connected to the first end of the bypass path, and the second end of the bypass path ends at or within the sidewall of the neck region. The bypass path is configured to provide a fluid flow path to the needle alternate to a first path upon a build-up of pressure for fluid flow in the vial assembly, and the first path is directly disposed in the vial assembly between the particulate region and the neck region.

In another embodiment, a vial assembly system comprises a vial assembly and a needle. The vial assembly comprises a septum, a neck region, and a particulate region for containing particulates comprising a bypass valve, the neck region comprising a top, a bottom, and a sidewall disposed therebetween, and the needle comprises a port and a tip distal of the port, the tip configured to puncture the septum of the vial assembly disposed at the bottom of the neck region. The septum is configured to receive the needle such that the port is disposed in the neck region of the vial assembly. The bypass valve is connected to a bypass path including a first end and a second end. The bypass valve is connected to the first end of the bypass path, and the second end of the bypass path ends at or within the sidewall of the neck region. The bypass path is configured to provide a fluid flow path to the needle alternate to a first path upon a build-up of pressure for fluid flow in the vial assembly, and the first path is directly disposed in the vial assembly between the particulate region and the neck region. The bypass path comprises a tubing disposed between the bypass valve and the sidewall of the neck region, and the tubing of the bypass path is radially disposed with respect to a longitudinal axis of the vial assembly.

In yet another embodiments, a method of operating a vial assembly system comprises receiving a needle in a septum of a vial assembly of the vial assembly system such that a port of the needle is disposed in a neck region of the vial assembly. The vial assembly further comprises a particulate region comprising a bypass valve, the neck region comprising a top, a bottom, and a sidewall disposed therebetween, the needle further comprising a tip distal of the port, the tip configured to puncture the septum of the vial assembly disposed at the bottom of the neck region. The method further comprises monitoring a pressure build-up in the vial assembly to determine whether the pressure build-up exceeds a threshold, and, when the pressure build-up that is monitored exceeds the threshold, alternating a fluid flow path from a first path to a bypass path. The bypass path includes a first end and a second end, the bypass valve is connected to the first end of the bypass path, the second end of the bypass path ends at or within the sidewall of the neck region, and the first path is directly disposed in the vial assembly between the particulate region and the neck region.

These and additional features provided by the embodiments described herein will be more fully understood in view of the following detailed description, in conjunction with the drawings.

### BRIEF DESCRIPTION OF THE DRAWINGS

FIG. 1 is a perspective view of a delivery device including a protective shield and a vial sled according to one or more embodiments shown and described herein;

FIG. 2 is a cross-sectional view of the vial sled of FIG. 1 according to one or more embodiments shown and described herein, the cross-section along line 2-2 of FIG. 1;

FIG. 3 is a perspective view of a vial assembly including an engagement head according to one or more embodiments shown and described herein;

FIG. 4 is a partial cross-sectional view of the vial assembly of FIG. 4, the cross-section taken along line 4-4 of FIG. 3;

FIG. 5 is a perspective view of the vial sled of FIG. 1 with the vial assembly of FIG. 3 received therein, with a series of delivery lines coupled to the vial sled, according to one or more embodiments shown and described herein;

FIG. 6 is a schematic side view of a vial assembly system that is prefilled with a particulate for delivery in a non-flow position, according to one or more embodiments shown and described herein;

FIG. 7 is a schematic side view of the vial assembly system of FIG. 6 in a bypass path flow position in which fluid including the particulate is directed along a bypass path to bypass a high concentration of underlying particulate in a vial assembly; and

FIG. 8 is a schematic side view of the vial assembly system of FIG. 6 in a first path flow position in which fluid including the particulate is directed along a first path disposed between a particulate and neck region of the vial assembly absent a high concentrate of underlying particulate in the vial assembly.

### DETAILED DESCRIPTION

Reference will now be made in detail to various embodiments of delivery devices for administering radioactive compounds to a patient, examples of which are illustrated in the accompanying drawings. Whenever possible, the same reference numerals will be used throughout the drawings to refer to the same or like parts. Directional terms as used herein-for example up, down, right, left, front, back, top, bottom, distal, and proximal-are made only with reference to the figures as drawn and are not intended to imply absolute orientation.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

Unless otherwise defined, all technical and scientific terms used herein have the same meaning as commonly understood by one of ordinary skill in the art to which the disclosure belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

As used herein, the terms “horizontal,” “vertical,” “distal” and “proximal” are relative terms only, are indicative of a general relative orientation only, and do not necessarily indicate perpendicularity. These terms also may be used for convenience to refer to orientations used in the figures, which orientations are used as a matter of convention only and are not intended as characteristic of the devices shown. The present disclosure and the embodiments thereof to be described herein may be used in any desired orientation. Moreover, horizontal and vertical walls need generally only be intersecting walls, and need not be perpendicular. As used herein, the singular forms “a,” “an” and “the” include plural referents unless the context clearly dictates otherwise. Thus, for example, reference to “a” component includes aspects having two or more such components, unless the context clearly indicates otherwise.

In embodiments described herein, a particulate material delivery assembly may include a radioembolization delivery device. A radioembolization delivery device comprises a medical device configured to deliver radioactive compounds to a treatment area within a patient's body in procedures such as transarterial radioembolization. The radioactive compounds may be a mixed solution of saline and radioactive microspheres (i.e., a particulate 660) mixed in a vial of a vial assembly. The needle may include one or more ports as an outlet to inject fluid (i.e., saline), such as from a syringe or catheter line, into a vial including the radioactive microspheres to generate the mixed solution 662 and as an inlet to deliver the mixed solution to the patient.

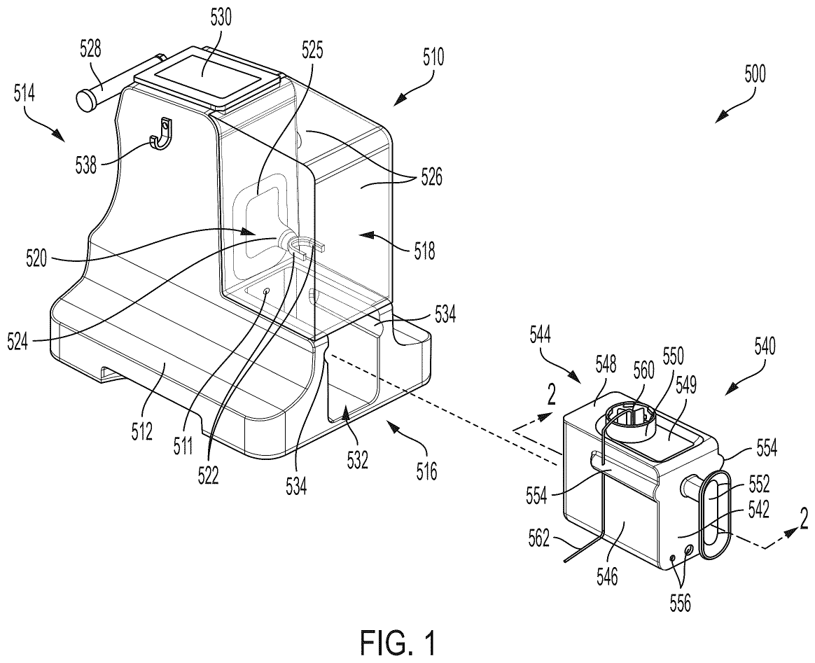

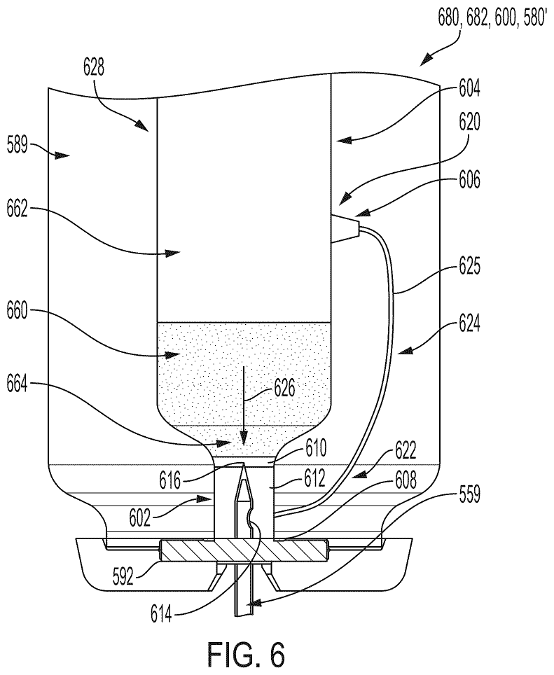

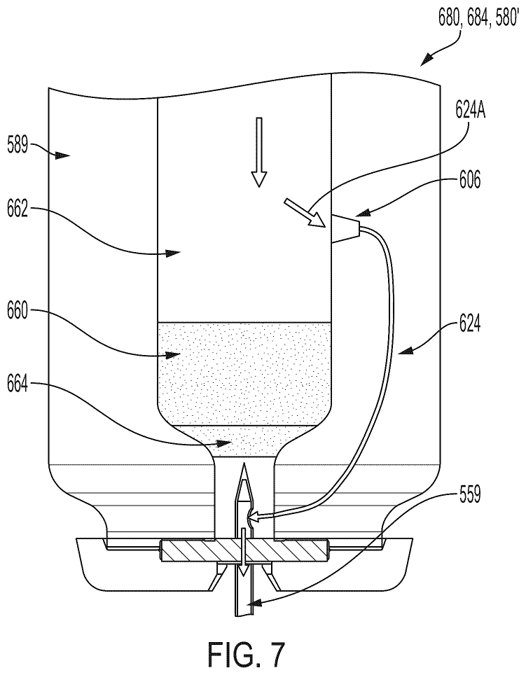

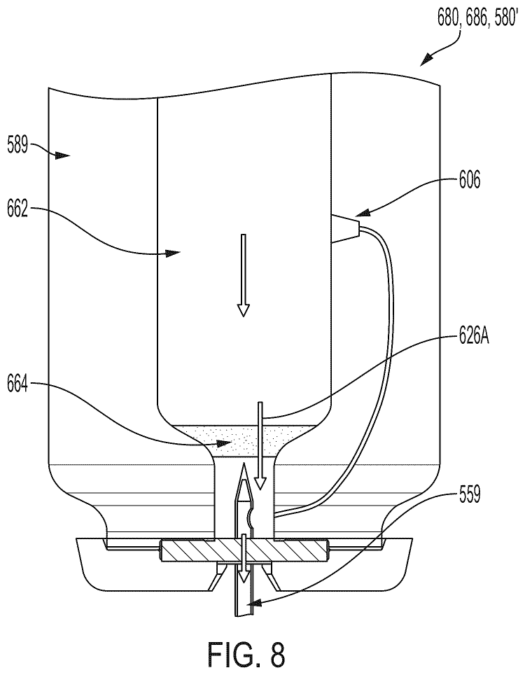

FIGS. 1-5 described below are directed to an embodiment of a delivery device 500 to deliver a particulate 660, and FIGS. 6-8 described in greater detail further below are directed to embodiments of one or more vial assembly system components of the delivery device 500 as described herein that assist with delivery of the particulate 660 within a mixed solution 662 as described herein to a patient. As will be described in greater detail below, FIG. 6 illustrates a vial assembly system 600 including a vial assembly 580′ such as a syringe assembly prefilled with embolic particles as the particulate 660 to be delivered as a concentration of particles via a mixed solution 662 that is compatible with a fluid path through, for example, a needle, tubing, catheter, or like delivery component, as also shown in FIG. 7-8. The vial assembly 580′ may include a concentration regulator 664, described in greater detail below, that may allow an appropriate concentration of particulate 660 within and with the mixed solution 662 through to the fluid path. If a bolus of a particulate 660 is too large for the fluid path and associated delivery component, either due to potential improper or insufficient mixing of the mixed solution 662 or settling of the particulate 660 due to gravity, the particulate 660 may clog the microcatheter and prevent administration. Thus, if the concentration of particulate 660 is too high compared to a threshold and may likely clog the fluid path delivery component such as a tubing, the concentration regulator 664 may restrict flow and allow the remaining low concentration fluid as overlying mixed solution 662 (over underlying suspended particulate 660) to bypass the concentration regulator 664 via a pressure sensitive unidirectional flow path such as through a bypass valve 606 as described herein. The bypass valve 606 may be triggered by a pressure sensor component or may be otherwise configured to open when a pressure exerted on the bypass valve 606 exceeds a threshold value.

In some embodiments, as described in greater detail below, the delivery device 500 is a radioembolization delivery device, the particulate 660 is a plurality of radioembolization beads, the fluid is a saline solution, and the resulting mixed fluid (e.g., the mixed solution 662) is a radioembolization beads-saline solution. The needle 559 may be configured to deliver the radioembolization beads-saline solution as the mixed fluid solution through the radioembolization delivery device, such as upon actuation of the vial engagement mechanism 520 in the positive pressure direction. In some embodiments, the fluid is a contrast-saline solution including a contrast agent, and the resulting mixed fluid (e.g., the mixed solution 662) is a radioembolization beads-contrast-saline solution. The needle 559 may be configured to deliver the radioembolization beads-contrast-saline solution as the mixed solution 662 through the radioembolization delivery device. In some embodiments, the delivery device 500 is a chemoembolization delivery device, the particulate 660 is a plurality of chemoembolization beads, and the mixed solution 662 is a beads-saline solution or a beads-contrast-saline solution.

### I. Mechanical Delivery Device with Removable Sled Assembly

FIGS. 1-5 show an embodiment of a delivery device 500 that is configured and operable to deliver a radioactive material (e.g., radioembolizing beads) while reducing radioactive emissions during use of the delivery device 500. The delivery device 500 may operate as described in International PCT App. No. PCT/2019/033001, filed May 17, 2019, the entirety of which is incorporated herein, except with respect to vial assembly system delivery flow components including a bypass path as described in greater detail below with respect to FIGS. 6-8 and in one or more embodiments herein.

Referring initially to FIG. 1, the delivery device 500 comprises a console assembly 510, which includes a console. The delivery device 500 may include a sled assembly 540 that is operable to transition between a coupled state and decoupled state relative to the console assembly 510. The console assembly 510 of the delivery device 500 comprises a base 512 defined by and extending between a proximal end 514 and a distal end 516. The proximal end 514 of the base 512 includes a handle (delivery handle) 528 movably coupled to the console assembly 510 and an interface display 530 positioned on the console assembly 510.

The proximal end 514 of the base 512 further includes an attachment device 538 that is configured to securely retain an external device to the base 512 of the console assembly 510. The attachment device 538 is operable to facilitate an attachment of a complimentary device to the console assembly 510 for use with the delivery device 500 during a procedure.

Still referring to FIG. 1, the distal end 516 of the console assembly 510 defines a vial containment region 518 that is sized and shaped to receive a vial assembly 580 therein, as will be described in greater detail herein. The console assembly 510 further includes a vial engagement mechanism 520 extending from the base 512 adjacent to the distal end 516. In particular, the vial engagement mechanism 520 extends laterally outward from the base 512 of the console assembly 510 toward the distal end 516. The vial engagement mechanism 520 is positioned within the vial containment region 518 of the console assembly 510 and is movably coupled to the handle 528. In particular, the handle 528 of the console assembly 510 is operable to move, and in particular translate, the vial engagement mechanism 520 within the vial containment region 518 in response to an actuation of the handle 528.

The console assembly 510 includes a mechanical assembly disposed within the base 512 that is configured and operable to convert a manual motion of the handle 528 to a corresponding linear displacement of the vial engagement mechanism 520. In the present example, the mechanical assembly is coupled to the handle 528 and the vial engagement mechanism 520 such that selective actuation of the handle 528 at the proximal end 514 causes a simultaneous actuation of the vial engagement mechanism 520 at the distal end 516.

The sled cavity 532 is sized and shaped to receive the sled assembly 540 therein. As will be described in greater detail herein, the sled assembly 540 is configured to store and administer therapeutic particles (e.g., radioactive beads, microspheres, medium) therethrough. In particular, the sled assembly 540 is configured to partially receive a vial assembly 580 therein for administering the therapeutic particles from the delivery device 500 and to a patient during a procedure.

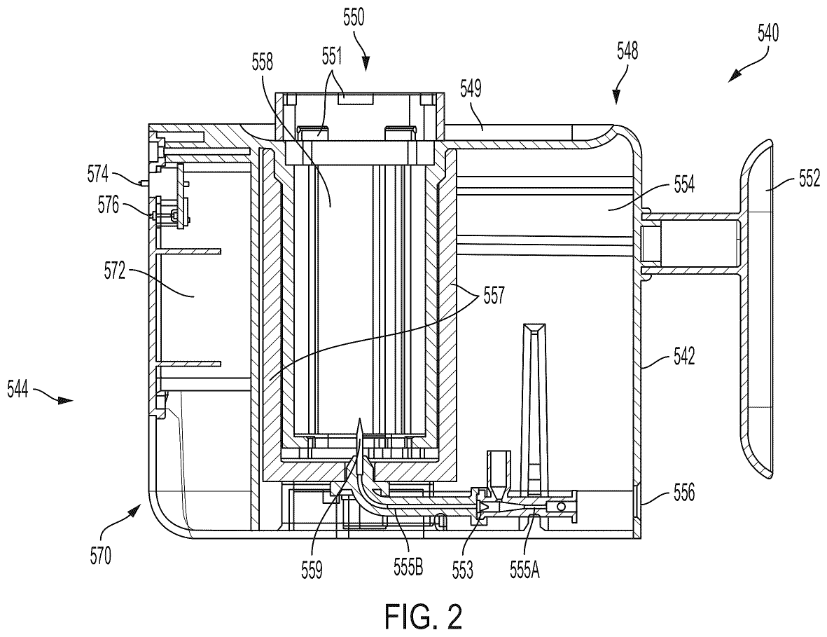

In embodiments, and referring to FIG. 2, a flow sensor of the delivery device 500 may be positioned in-line with the tubing set of the delivery device 500, and in particular the needle 559, the manifolds 555A, 555B, and/or one or more of the ports 556, and may be configured to measure an amount of fluid (e.g., suspension liquid after the therapeutic particles have effectively mixed with the fluid medium) that passes thereby. Referring back to FIG. 1, the vial engagement mechanism 520 comprises a pair of lever arms 522 extending outwardly from a neck 524 of the vial engagement mechanism 520, with the neck 524 extending laterally outward from the base 512 of the console assembly 510. The neck 524 of the vial engagement mechanism 520 is disposed within a protective cover 525 such that only the pair of lever arms 522 of the vial engagement mechanism 520 extends through the protective cover 525. The protective cover 525 is operable to shield one or more internal components of the console assembly 510 from an exterior of the console assembly 510, and in particular from the vial containment region 518.

The pair of lever arms 522 is simultaneously movable with the neck 524 of the vial engagement mechanism 520 in response to an actuation of the handle 528 of the console assembly 510. Further, the pair of lever arms 522 are fixed relative to one another such that a spacing formed between the pair of lever arms 522 is relatively fixed. The pair of lever arms 522 of the vial engagement mechanism 520 is configured to securely engage the vial assembly 580 therebetween, and in particular within the spacing formed by the pair of lever arms 522. Accordingly, the vial engagement mechanism 520 is operable to securely attach the vial assembly 580 to the console assembly 510 at the vial containment region 518. Although the vial engagement mechanism 520 is shown and described herein as including a pair of lever arms 522, it should be understood that the vial engagement mechanism 520 may include various other structural configurations suitable for engaging the vial assembly 580. In a non-limiting example, the vial engagement mechanism 520 may include one or more magnets configured to engage with one or more corresponding magnets on the vial assembly.

Still referring to FIG. 1, the console assembly 510 further includes a safety shield 526 secured to the distal end 516 of the base 512 along the vial containment region 518. In particular, the safety shield 526 is a protective covering that is sized and shaped to enclose the vial containment region 518 of the console assembly 510 when secured thereon. The safety shield 526 is selectively attachable to the distal end 516 of the base 512 and is formed of a material that is configured to inhibit radioactive emissions from one or more radioactive doses stored within the vial containment region 518.

The distal end 516 of the console assembly 510 further includes a sled cavity 532 that is sized and shaped to receive the sled assembly 540 therein. The sled cavity 532 includes one or more or a pair of alignment features 534 extending therein, with the alignment features 534 sized and shaped to correspond with complimentary alignment features of the sled assembly 540 (e.g., alignment ribs 554) to thereby facilitate a coupling of the sled assembly 540 with the base 512 of the console assembly 510 within the sled cavity 532.

Still referring to FIG. 1, the sled assembly 540 is configured to partially receive a vial assembly 580 therein for administering therapeutic particles (e.g., radioactive fluid medium) from the delivery device 500 and to a patient. In particular, the sled assembly 540 comprises a distal end 542 and a proximal end 544 with a pair of sidewalls 546 extending therebetween. The distal end 542 of the sled assembly 540 includes a handle 552 extending proximally therefrom. The handle 552 is configured to facilitate movement of the sled assembly 540, and in particular, an insertion of the sled assembly 540 into the sled cavity 532 of the console assembly 510. The distal end 542 further includes one or more ports 556 for coupling one or more delivery lines (i.e., tubing) to the sled assembly 540. With the one or more delivery lines further be coupled to one or more external devices at an end of the line opposite of the ports 556, the ports 556 effectively serve to fluidly couple the sled assembly 540 to the one or more external devices via the delivery lines connected thereto. The pair of sidewalls 546 of the sled assembly 540 includes at least one alignment rib 554 extending laterally outward therefrom, where the alignment ribs 554 are sized and shaped to correspond with and mate to the pair of alignment features 534 of the console assembly 510. Accordingly, the pair of alignment ribs 554 are configured to facilitate an alignment and engagement of the sled assembly 540 with the console assembly 510 when the proximal end 544 is slidably received within the sled cavity 532 of the base 512.

The sled assembly 540 further includes a top surface 548 extending from the distal end 542 and the proximal end 544 and positioned between the pair of sidewalls 546. The top surface 548 of the sled assembly includes a recessed region 549 and a locking system 550. The recessed region 549 is sized and shaped to form a recess and/or cavity along the top surface 548, where the recessed region 549 is capable of receiving and/or collecting various materials therein, including, for example, leaks of various fluid media during use of the delivery device 500. The locking system 550 of the sled assembly 540 forms an opening along the top surface 548 that is sized and shaped to receive one or more devices therein, such as a priming assembly 560 and a vial assembly 580. In some embodiments, the sled assembly 540 comes preloaded with the priming assembly 560 disposed within the locking system 550. The priming assembly 560 includes a priming line 562 extending outwardly from the locking system 550 of the sled assembly 540. The priming assembly 560 connects the priming line 562 to needle 559 and manifolds 555A and 555B and serves to purge the delivery device 500, including the manifolds 555A and 555B, of air prior to utilizing the delivery device 500 in a procedure.

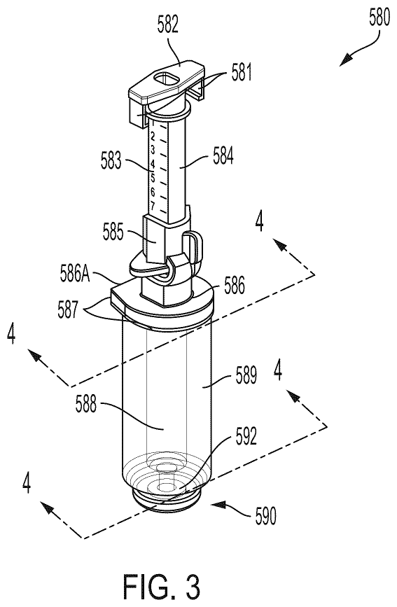

Referring now to FIG. 2, the locking system 550 includes an annular array of projections 551 extending outwardly therefrom, and in particular, extending laterally into the aperture formed by the locking system 550 along the top surface 548. The annular array of projections 551 are formed within an inner perimeter of the locking system 550 and extend along at least two sequentially-arranged rows. In embodiments, a single row may be used. The annular array of projections 551 included in the locking system 550 are configured to engage a corresponding locking feature 586 of the vial assembly 580 (See FIG. 3) to thereby securely fasten the vial assembly 580 to the sled assembly 540. It should be understood that the multiple rows of projections 551 of the locking system 550 serve to provide a double-locking system to ensure the sled assembly 540, and in particular a needle 559 of the sled assembly 540, is securely maintained through a septum 592 of the vial assembly 580 (See FIG. 3) during use of the delivery device 500 in a procedure.

The sled assembly 540 further includes a vial chamber 558 that is sized and shaped to receive the priming assembly 560 and the vial assembly 580 therein, respectively. In other words, the vial chamber 558 is sized to individually receive both the priming assembly 560 and the vial assembly 580 separate from one another. The vial chamber 558 is encapsulated around a protective chamber or shield 557 disposed about the vial chamber 558. The protective shield 557 is formed of a material configured to inhibit radioactive emissions from extending outwardly from the vial chamber 558, such as, for example, a metal or plastic. Additionally, the sled assembly 540 includes a needle extending through the protective shield 557 and into the vial chamber 558 along a bottom end of the vial chamber 558. The needle 559 is fixedly secured relative to the vial chamber 558 such that any devices received through the aperture of the locking system 550 and into the vial chamber 558 are to encounter and interact with the needle 559 (e.g., the priming assembly 560, the vial assembly 580, and the like).

Still referring to FIG. 2, the needle 559 is coupled to a distal manifold 555A and a proximal manifold 555B disposed within the sled assembly 540, and in particular the manifold 555A, 555B is positioned beneath the vial chamber 558 and the protective shield 557. The proximal manifold 555B is fluidly coupled to the needle 559 and the distal manifold 555A is fluidly couplable to one or more delivery lines via the one or more ports 556 of the sled assembly 540. The proximal manifold 555B is in fluid communication with the distal manifold 555A through a one-way check valve 553 disposed therebetween.

Accordingly, the proximal manifold 555B is in fluid communication with the one or more ports 556 via the distal manifold 555A, however, the one or more ports 556 are not in fluid communication with the proximal manifold 555B due to a position of the one-way check valve 553 disposed between the manifolds 555A, 555B. Thus, the needle 559 is in fluid communication with the one or more delivery lines and/or devices coupled to the sled assembly 540 at the one or more ports 556 via the manifolds 555A, 555B secured therebetween. The one or more ports 556 of the sled assembly 540 may be coupled to a bag (e.g., saline bag), a syringe, a catheter, and/or the like via one or more delivery lines coupled thereto. In other embodiments, the needle 559 may be a cannula, catheter, or similar mechanism through which to inject and receive fluid and/or a solution as described herein.

Still referring to FIG. 2, the sled assembly 540 includes a removable battery pack 570 coupled to the sled assembly 540 along the proximal end 544. The removable battery pack 570 comprises a battery 572, electrical contacts 574, and a removable tab 576. The battery 572 of the delivery device 500 is isolated from one or more fluid paths and radiation sources due to a location of the battery 572 in the removable battery pack 570.

The electrical contacts 574 of the removable battery pack 570 extend outwardly from the removable battery pack 570 and are operable to contact against and interact with corresponding electrical contacts 511 of the console assembly 510 (See FIG. 1) when the sled assembly 540 is coupled to the base 512 at the sled cavity 532. Accordingly, the removable battery pack 570 is operable to provide electrical power to the delivery device 500, and in particular the console assembly 510, when the sled assembly 540 is coupled to the console assembly 510.

Additionally, as will be described in greater detail herein, in some embodiments the locking system 550 may include at least one planar wall relative to a remaining circular orientation of the locking system 550. In this instance, an aperture formed by the locking system 550 through the top surface 548 of the sled assembly 540 is irregularly-shaped, rather than circularly-shaped as shown and described above. In this instance, the vial assembly 580 includes a locking feature 586 that has a shape and size that corresponds to the locking system 550, and in particular the at least one planar wall such that the vial assembly 580 is received within the sled assembly 540 only when an orientation of the vial assembly 580 corresponds with an alignment of the locking feature 586 and the locking system 550. In other words, a corresponding planar wall 586A of the locking feature 586 (See FIG. 3) must be aligned with the planar wall of the locking system 550 for the vial assembly 580 to be receivable within an aperture formed by the locking system 550 of the sled assembly 540.

Referring now to FIG. 3, the vial assembly 580 of the delivery device 500 is depicted. The vial assembly 580 comprises an engagement head 582, a plunger 584, a locking feature 586, and a vial body 589. In particular, the engagement head 582 of the vial assembly 580 is positioned at a terminal end of the plunger 584 opposite of the locking feature 586 and the vial body 589. The engagement head 582 includes a pair of arms 581 extending laterally outward relative to a longitudinal length of the plunger 584 extending downwardly therefrom. In the present example, the engagement head 582 is integrally formed with the plunger 584, however, it should be understood that in other embodiments the engagement head 582 and the plunger 584 may be separate features fastened thereto. In either instance, the engagement head 582 and the plunger 584 is movable relative to the locking feature 586 and the vial body 589 such that the engagement head 582 and the plunger 584 are slidably translatable through the locking feature 586 and the vial body 589. In particular, as will be described in greater detail herein, the plunger 584 may translate into and out of an internal chamber 588 of the vial body 589 in response to a linear translation of the vial engagement mechanism 520 when the engagement head 582 is secured to the pair of lever arms 522.

The plunger 584 includes a plurality of indicia and/or markings 583 positioned along a longitudinal length of the plunger 584. The plurality of markings 583 is indicative of a relative extension of the engagement head 582 and the plunger 584 from the locking feature 586 and the vial body 589. As briefly noted above, the engagement head 582 is configured to attach the vial assembly 580 to the vial engagement mechanism 520. In particular, the pair of arms 581 of the engagement head 582 are sized and shaped to couple with the pair of lever arms 522 of the vial engagement mechanism 520 when the vial assembly 580 is received within the sled assembly 540 and the sled assembly is inserted into the sled cavity 532 of the console assembly 510. As will be described in greater detail herein, the pair of lever arms 522 are received between the pair of arms 581 of the engagement head 582 and the plunger 584 in response to a predetermined translation force applied to the vial engagement mechanism 520. The engagement head 582 and the plunger 584 may be formed of various materials, including, but not limited to, a metal, plastic, and/or the like.

Still referring to FIG. 3, the vial assembly 580 further includes a safety tab 585 coupled to the plunger 584 relatively above the locking feature 586 and below the engagement head 582 such that the safety tab 585 is positioned along the longitudinal length of the plunger 584. The safety tab 585 may be formed of various materials, such as, for example, a plastic, and is preassembled onto the vial assembly 580 prior to a use of the delivery device 500. The safety tab 585 is removably fastened to the plunger 584 and inhibits the plunger 584 from translating relative to the vial body 589. In particular, the safety tab 585 abuts against the locking feature 586 in response to an application of linear force onto the plunger 584 to translate the plunger 584 relatively downward into the vial body 589. In this instance, the safety tab 585 is configured to inhibit an inadvertent movement of the plunger 584, and in response, an inadvertent delivery of a fluid media stored within the internal chamber 588 of the vial body 589 (e.g., therapeutic particles, radioembolizing beads). As will be described in greater detail herein, the safety tab 585 is selectively disengaged from the plunger 584 in response to a coupling of the vial assembly 580 with the vial engagement mechanism 520, and in particular an engagement of the pair of lever arms 522 with the engagement head 582.

Referring back to FIG. 3, the locking feature 586 extends about a top end of the vial body 589. In the present example, the locking feature 586 of the vial assembly 580 comprises a bushing that defines a lateral edge 587 extending laterally outward along an outer perimeter of the locking feature 586. The lateral edge 587 of the locking feature 586 is sized and shaped to engage the annular array of projections 551 of the locking system 550 when the vial assembly 580 is received within the vial chamber 558 of the sled assembly 540. As will be described in greater detail herein, the locking feature 586, and in particular the lateral edge 587 of the locking feature 586, is configured to securely fasten the vial assembly 580 to the locking system 550 to inhibit removal of the vial body 589 from the vial chamber 558 of the sled assembly 540 during use of the delivery device 500 in a procedure. In some embodiments, as briefly described above, the locking feature 586 includes at least one planar wall 586A such that the locking feature 586 comprises an irregular-profile. The at least one planar wall 586A is configured to correspond to the planar wall 550A of the locking system 550 such that an alignment of the planar walls 550A, 586A is required for the vial assembly 580 to be received through an aperture formed by the locking system 550.

Still referring to FIG. 3, the vial body 589 extends downwardly relative from the locking feature 586 and has a longitudinal length that is sized to receive at least a portion of a longitudinal length of the plunger 584 therein. Accordingly, in some embodiments a longitudinal length of the plunger 584 exceed a longitudinal length of the vial body 589 such that a translation of the plunger 584 into the internal chamber 588 of the vial body 589 causes a fluid media stored therein to be transferred outward from the vial body 589. As will be described in greater detail herein, a translation of the plunger 584 through the internal chamber 588 of the vial body 589 provides for an administration of a fluid media stored within the vial body 589 outward from the vial assembly 580. The vial body 589 may be formed of various materials, including, for example, a thermoplastic polymer, copolyester, polycarbonate, a biocompatible plastic, polysulfone, ceramics, metals, and/or the like.

The vial body 589 is of the present example is formed of a material that is configured to inhibit radioactive emissions from a fluid media stored within the internal chamber 588 of the vial body 589. For example, the vial body 589 may be formed of a plastic, such as polycarbonate, and have a width. A density and material composition of the vial body 589 may collectively inhibit beta radiation emission from electron particles stored within the internal chamber 588. In the present example, a chemical composition of the plastic of the vial body 589, along with the 9 mm wall thickness, provides a plurality of atoms disposed within the vial body 589 that are capable of encountering the electron particles generating beta radiation and reducing an emission of said radiation from the vial assembly 580. Accordingly, the vial assembly 580 allows an operator to handle the radioactive material stored within the vial body 589 without being exposed to beta radiation. It should be understood that various other materials and/or wall sections may be incorporated in the vial body 589 of the vial assembly 580 in other embodiments without departing from the scope of the present disclosure.

Still referring to FIG. 3, the vial body 589 of the vial assembly 580 is sealed at a first terminal end 598 by the locking feature 586. The vial assembly 580 further includes a cap 590 positioned at an opposing, terminal end of the vial body 589 opposite of the locking feature 586, such that the cap 590 seals a second terminal end of the vial body 589 of the vial assembly 580. Additionally, the vial assembly 580 includes a septum 592 positioned adjacent to the cap 590 and in fluid communication with a terminal end of the vial body 589 opposite of the locking feature 586. The septum 592 forms a seal against a terminal end of the vial body 589 and the cap 590 retains the septum 592 therein. The septum 592 may be formed of various materials, including, for example, an elastomer, silicon, bromobutyl elastomer, rubber, urethanes, and/or the like. The septum 592 is configured to provide an air-tight seal for the vial body 589 to thereby inhibit a release of a fluid media stored therein (e.g., radioembolizing beads). As will be described in greater detail herein, the septum 592 of the vial assembly 580 is configured to be punctured by the needle 559 of the sled assembly 540 when the vial assembly 580 is received within the vial chamber 558, thereby establishing fluid communication between the vial body 589 and the sled assembly 540. In other embodiments, the septum 592 may be omitted entirely for an alternative device, such as, for example, a valve system, needle injection port, and/or the like.

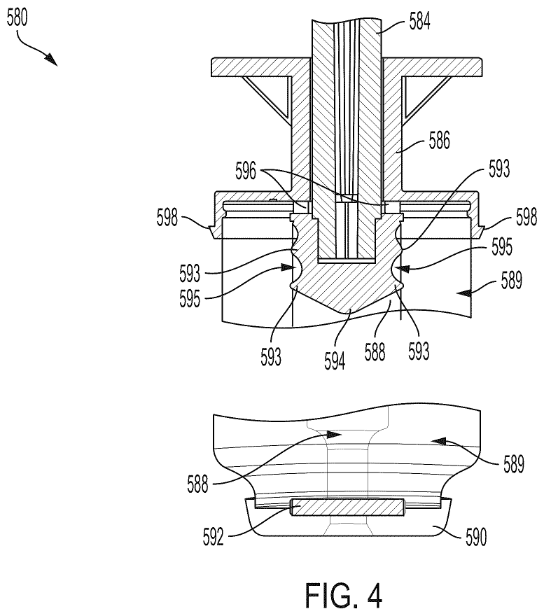

Referring to FIG. 4, the vial assembly 580 further includes a stopper 594 fixedly coupled to a terminal end of the plunger 584 opposite of the engagement head 582. In this instance, with the plunger 584 coupled to, and slidably translatable through, the internal chamber 588 of the vial body 589, the stopper 594 is effectively disposed within the vial body 589. Accordingly, it should be understood that the stopper 594 is sized and shaped in accordance with a size (e.g., a diameter) of the internal chamber 588 of the vial body 589. The stopper 594 is secured to the plunger 584 such that the stopper 594 is slidably translatable through the vial body 589 in response to a translation of the plunger 584 through the vial body 589. The stopper 594 is defined by two or more ribs 593 extending laterally outward and one or more troughs 595 defined between at least two ribs 593.

The stopper 594 is configured to form a liquid-seal against the internal chamber 588 of the vial body 589, and may be formed of a various polymers with a predetermined viscoelasticity. For example, in some embodiments the stopper 594 is formed of an elastomer, silicone, rubber, urethane, plastic, polyethylene, polypropylene, and/or the like. In this instance, the stopper 594 is operable to inhibit a fluid media stored within the vial body 589 from extending (i.e., leaking) past the stopper 594 and out of the vial body 589. In particular, the two or more ribs 593 of the stopper 594 abut against, and form a seal along, the internal chamber 588 of the vial body 589 to thereby inhibit a fluid media from passing beyond the ribs 593. The one or more troughs 595 formed between the two or more ribs 593 of the stopper 594 are configured to receive, and more specifically capture, any fluid media that may inadvertently extend (i.e., leak) beyond the ribs 593 of the stopper 594. Accordingly, the one or more troughs 595 serve as a safety mechanism of the vial assembly 580 to ensure a fluid media is maintained within the vial body 589 and not exposed beyond the vial assembly 580.

Still referring to FIG. 4, the two or more ribs 593 of the stopper 594 are additionally configured to push a fluid media stored within the vial body 589 in one or more directions therein (e.g., toward the cap 590) in response to a translation of the plunger 584. With the ribs 593 of the stopper 594 pressed against the internal chamber 588 of the vial body 589, translation of the plunger 584 provides for a translation of the ribs 593 against and along the internal chamber 588 of the vial body 589 such that any fluid media located in front (i.e., beneath) of the stopper 594 is effectively redirected within the vial body 589 in a direction of travel of the plunger 584 and the stopper 594. The vial assembly 580 further includes an annular washer 596 disposed within the vial body 589. In particular, the annular washer 596 is securely fixed to the plunger 584 adjacent to the stopper 594, which is secured to the plunger 584 at a terminal end opposite of the engagement head 582. Accordingly, the annular washer 596 is secured to the plunger 584 and disposed within the vial body 589 adjacent to the stopper 594. With the annular washer 596 secured to the plunger 584 adjacent to the stopper 594, the annular washer 596 is effectively disposed within the vial body 589.

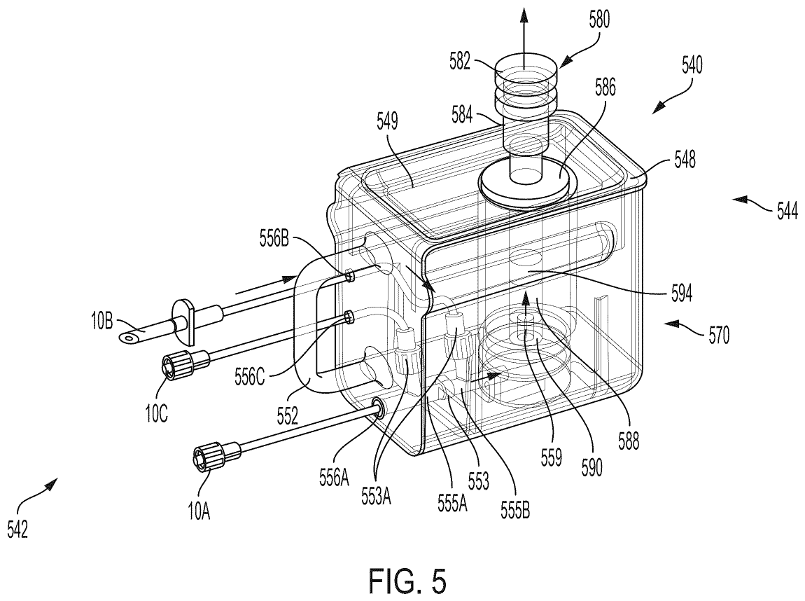

Referring now to FIG. 5, in response to determining that the battery 572 contains or other power source provides a sufficient amount of power, one or more delivery lines are coupled to the sled assembly 540 via the one or more ports 556. In particular, a dose delivery line 10A is coupled to the sled assembly 540 at a delivery port 556A, a contrast line 10B is coupled to the sled assembly 540 at a contrast port 556B, and a flushing line 10C is coupled to the sled assembly 540 at a flushing port 556C. An opposing end of the dose delivery line 10A is initially coupled to a fluid reservoir, such as, for example, a collection bowl. As will be described in greater detail herein, the dose delivery line 10A may be subsequently coupled to an external device, such as a catheter, once the sled assembly 540 has been effectively primed by a fluid medium via the contrast line 10B. An opposing end of the flushing line 10C is coupled to an external device, such as, for example, a syringe. With both the dose delivery line 10A and the flushing line 10C coupled to the sled assembly 540, the sled assembly 540 is flushed with a fluid medium (e.g., saline) from the syringe coupled to the flushing line 10C. In this instance, the fluid medium is injected through the flushing line 10C, into the distal manifold 555A of the sled assembly 540, and out of the sled assembly 540 through the dose delivery line 10A. Accordingly, the fluid medium is ultimately received at the collection bowl and disposed thereat by the dose delivery line 10A.

With the distal manifold 555A of the sled assembly 540 separated from the proximal manifold 555B by the one-way valve 553 disposed therebetween, the fluid medium flushed through the distal manifold 555A from the syringe (via the flushing port 556C) is prevented from passing through the proximal manifold 555B and the needle 559 coupled thereto. Rather, the fluid medium injected from the syringe and through the flushing line 10C is received at the flushing port 556C, passed through the distal manifold 555A in fluid communication with the flushing port 556C, and redirected by the one-way valve 553 towards the dose delivery port 556A that is coupled to the dose delivery line 10A. In this instance, the dose delivery line 10A receives and transfers the fluid medium to the collection bowl coupled thereto, such that the fluid medium is not directed beyond the one-way valve 553 and into the proximal manifold 555B that is in fluid communication with the needle 559.

The contrast line 10B is coupled to the sled assembly 540 at a contrast port 556B. An opposing end of the contrast line 10B is coupled to a fluid medium supply, such as, for example, a bag secured to the console assembly 510 via the attachment device 538. In the present example, the bag is a saline bag such that the fluid medium stored therein is saline. In this instance, with the sled assembly 540 including the priming assembly 560 positioned within the vial chamber 558 and the needle end 568 in fluid communication with the needle 559, a syringe is fluidly coupled to the priming line 562 of the priming assembly 560 and a plunger of the syringe is drawn back to pull saline through the contrast line 10B, the contrast port 556B, the sled assembly 540, the priming line 562 and into the syringe from the saline bag. The plunger of the syringe is thereafter pushed inwards to transfer the extracted saline back through the priming line 562, the central body 564, the elongated shaft 566, and the needle end of the priming assembly 560 such that the saline is received into the needle 559 of the sled assembly 540. Accordingly, the manifolds 555A, 555B of the sled assembly 540 are effectively primed with the saline from the syringe as the needle 559 that received the saline from the priming assembly 560 is in fluid communication with the manifolds 555A, 555B. With the manifolds 555A, 555B in further fluid communication with the dose delivery line 10A via the delivery port 556A, the saline is effectively distributed to the collection bowl coupled thereto.

Referring now to FIG. 5, the sled assembly 540 is coupled to one or more external devices via the one or more ports 556. In particular, the sled assembly 540 is fluidly coupled to a catheter (e.g., microcatheter) via the dose delivery line 10A that is coupled to the delivery port 556A of the sled assembly 540. In this instance, the catheter is in fluid communication with the sled assembly 540 via the dose delivery line 10A. Further, the sled assembly 540 is fluidly coupled to a contrast source, such as, for example, a saline bag secured to the console assembly 510 via the attachment device 538 (See FIG. 1). The sled assembly 540 is in fluid communication with the saline bag via a contrast line 10B coupled to the contrast port 556B of the sled assembly 540. In this instance, the saline bag is in fluid communication with the sled assembly 540 via the contrast line 10B secured to the contrast port 556B.

The contrast port 556B is in fluid communication with the proximal manifold 555B while the delivery port 556A is in fluid communication with the distal manifold 555A. As will be described in greater detail herein, saline from the saline bag may be withdrawn through the needle 559 of the sled assembly 540 and into the vial body 589 of the vial assembly 580 as the contrast port 556B is coupled to the proximal manifold 555B, rather than the distal manifold 555A which is separated from the proximal manifold 555B by the one-way check valve 553 disposed therebetween.

Referring again to FIGS. 1 and 3, with the vial assembly 580 securely coupled to the sled assembly 540, the sled assembly 540 is coupled to the console assembly 510 by translating the distal end 542 of the sled assembly 540 toward and into the distal end 516 of the console assembly 510. In particular, the distal end 542 of the sled assembly 540 is directed into the sled cavity 532 of the console assembly 510 by aligning the alignment ribs 554 of the sled assembly 540 with the alignment features 534 of the console assembly 510. Once the proximal end 544 and the distal end 542 of the sled assembly 540 are fully seated within the sled cavity 532 of the console assembly 510, the electrical contacts 574 (FIG. 2) of the removable battery pack 570 interact with corresponding electrical contacts 511 (FIG. 1) of the console assembly 510. In this instance, power from the battery 572 is transmitted to the console assembly 510 via the electrical contacts 574, thereby activating the console assembly 510 of the delivery device 500. In this instance, the interface display 530 of the console assembly 510 is activated to display pertinent, real-time information relating to the delivery device 500 during a procedure.

Referring again to FIG. 5, as the vial engagement mechanism 520 and the plunger 584 are simultaneously translated within the vial containment region 518, a negative pressure is generated within the internal chamber 588 of the vial body 589 due to a retraction of the stopper 594. In this instance, with the saline bag coupled to the sled assembly 540 via the contrast line 10B and the contrast port 556B, saline from the saline bag is pulled into the internal chamber 588 of the vial body 589 through the proximal manifold 555B and the needle 559. Accordingly, with the vial body 589 being preloaded with a radioactive fluid media (e.g., radioembolizing microspheres), the saline is effectively mixed with the radioactive fluid media within the vial body 589 as the plunger 584 is retracted from the internal chamber 588 and the negative pressure is generated through the delivery device 500.

The sled assembly 540 further includes one-way check valves 553A in-line with the contrast line 10B and the flushing line 10C. In particular, the one-way check valves 553A are configured to permit fluid communication from the contrast port 556B and the flushing port 556C into the manifolds 555A, 555B, and further configured to prevent fluid communication from the manifolds 555A, 555B to the contrast port 556B and the flushing port 556C. Accordingly, it should be understood that the dose delivered from the vial body 589 to the manifold 555A, 555B is incapable of being directed into the contrast line 10B or the flushing line 10C due to the one-way check valves 553A positioned therein. Thus, the dose is directed to the dose delivery port 556A and received at the catheter fluidly coupled thereto by the dose delivery line 10A. In other words, the one-way check valves 553A prevent a backflow of fluid into the sled assembly 540 and/or the vial assembly 580 coupled thereto.

### II. Vial Assembly Systems with Bypass Path Embodiments

As briefly noted above, the delivery device 500 described herein may include a vial assembly system 680 including a bypass path that may be used to effectively deliver the particulate 660 via the mixed solution 662, embodiments of which are described in greater detail below with respect to FIGS. 6-8. In embodiments, and referring to FIG. 6 illustrating the vial assembly system 680 prefilled with a particulate 660 for delivery in a non-flow position 682, the vial assembly system 680 includes a vial assembly 580′ and a needle 559. The vial assembly 580′ may include a vial body 589 including a septum 592, a neck region 602, and a particulate region 604. The neck region 602 may include a cylindrical shape, a conical shape, a tapered shape, or other suitable shape. The particulate region 604 may include a bypass valve 606. In embodiments, the bypass valve 606 may include a one-way valve, check valve, pop-off valve, or other suitable valve configured for one-way directional flow. The bypass valve 606 disposed in the particulate region 604 may be further disposed in a deadspace region 628 of the particulate region 604. The deadspace region 628 is defined as an area in which a plunger 584 (FIG. 5) of the vial assembly 580′ is unable to distally (i.e., in a downward direction toward the septum 592) plunge further into the area and thus cannot plunge into the deadspace region 628. In embodiments, the vial assembly 580′ may be a syringe assembly as described herein including a plunger 584 (FIG. 5), barrel, stopper, and needle 559, the concentration regulator 664 as described in greater detail further below, the bypass valve 606 (such as a one-way valve that may be a pop-off valve, duck bill valve, or the like), and a fluid path such as through a polyvinyl chloride (PVC) medical tubing connecting the bypass valve 606 to the needle while by-passing the concentration regulator 664.

The neck region 602 may include a top 610, a bottom 608, and a sidewall 612 disposed therebetween. The needle 559 may include a port 614 and a tip 616 distal of the port 614. The port 614 of the needle 559 may be configured as an outlet to inject fluid into the vial assembly 580′ and as an inlet to deliver a mixed particulate solution of fluid and particulate 660 (e.g., the mixed solution 662) from the vial assembly 580′. The tip 616 may be configured to puncture the septum 592 of the vial assembly 580′ disposed at the bottom 608 of the neck region 602. The septum 592 may be configured to receive the needle 559 such that the port 614 is disposed in the neck region 602 of the vial assembly 580′. The neck region 602 may be sized and shaped to receive both the tip 616 and the port 614 between the septum 592 and a concentration regulator 664, which is described in greater detail further below. The septum 592 may be configured to be disposed proximally adjacent to (upward relative to) the neck region 602, and the particulate region 604 may be disposed distally adjacent to (downward relative to) the neck region 602.

A bypass path 624 as shown includes a first end 620 and a second end 622. The bypass valve 606 is connected to the first end 620 of the bypass path 624. The second end 622 of the bypass path 624 ends at or within the sidewall 612 of the neck region 602. The bypass path 624 is configured to provide a fluid flow path via the bypass path 624 to the needle alternate to a first path 626 upon a build-up of pressure for fluid flow in the vial assembly 580. As shown, the first path 626 is directly disposed in the vial assembly 580′ between the particulate region 604 and the neck region 602. In embodiments, the first path 626 is directed along a longitudinal axis of the vial assembly 580′, and the bypass path 624 is radially disposed with respect to the longitudinal axis. The bypass path 624 may include a tubing 625 disposed between the bypass valve 606 and the sidewall 612 of the neck region 602. In embodiments, rather than the tubing 625, the bypass path 624 may be a needle or an alternate metal flow path.

In embodiments, the vial assembly system 680 may further include a concentration regulator 664 disposed between the neck region 602 and the particulate region 604. The concentration regulator 664 may be configured to restrict a concentration of particulates 660 to send along the first path 626 to the needle 559. The concentrator regulator 664 may be a mesh component. Alternatively or additionally, the concentrator regulator 664 may be a filter, membrane, or other suitable regulator component configured to restrict flow of a high concentration of particulates 660 that is above a concentration threshold.

The vial assembly 580′ may be installed into the delivery device 500 as described herein, and the plunger 584 (FIG. 5) may be pulled all of the way up to draw fluid into a vial of the vial assembly 580′ and suspend the particulate 660. The fluid may be prevented from going up the bypass path 624 due to the one-way bypass valve 606, and the fluid may be drawn through the concentration regulator 664 to suspend the particulate 660 in the mixed solution 662. If a user delays and the particulate 660 settle out of suspension as shown in FIG. 6, a large bolus of particulate 660 may accumulate on top of the concentration regulator 664. In this state, the user could push down on the plunger 584 and, once sufficient pressure is achieved, the one way bypass valve 606 may open and allow the low concentration fluid (including a lower particulate 660 concentration along with fluid) of the mixed solution 662 to flow out to the needle 559 along the bypass path 624 as shown in FIG. 7. Alternatively, in embodiments, the concentration regulator 664 may be used as a pressure sensor or another pressure sensor may be used to trigger the bypass valve 606 upon detection of a pressure above a threshold. The user may continue to administer the mixed solution 662 until the plunger 584 blocks the one way bypass valve 606. At this point, the user may again draw up the plunger 584 to a maximum position and re-suspend and administer the spheres of the particulate 660 in the mixed solution 662 as an appropriate concentration suitable for the first path 626 as shown in FIG. 8.

Thus, referring to FIG. 7, a bypass path flow position 684 is shown in which fluid including the particulate 660 as the mixed solution 662 is directed along the bypass path 624A, 624 to bypass a high concentration of underlying particulate 660 shown as unsuspended below the suspending mixed solution 662 in a vial assembly 580′. In embodiments, the bypass path 624 is configured to provide the fluid flow path to the needle 559 alternate to the first path 626 upon the build-up of pressure for fluid flow in the vial assembly 580′ when a build-up of particulates 660 occur on the concentration regulator 664.

In embodiments, both the concentration regulator 664 and the bypass valve 606 may be configured such that the particulate 660 does not get stuck in the mesh, membrane, filter, or the like, or valve features of the bypass valve 606. Additionally, holes in the concentration regulator 664 may be sized to allow the particulate 660 to pass through them but spaced such that only an appropriate and predetermined quantity can pass through at a time without the fluid path of the first path 626 clogging. The bypass valve 606 may be positioned above where a maximum concentration of particulate 660 would occur and sufficiently below a top of the plunger 584 stroke to allow for the particulate 660 to be re-suspended. The pressure required to open the bypass valve 606 may be such that the particulate 660 and the concentration regulator 664 aren't damaged. The maximum operating pressure of the particulate 660 and the concentration regulator 664 may depend on the type of particulate and concentration regulator used, and the maximum operating pressure can be determined by applied controlled predetermined pressures to both to observe the maximum pressure that the particulates and concentration regulator withstand wihtout being damaged. In embodiments, the bypassing fluid path as the bypass path 624 may be overmolded or made as a part of the syringe body of the vial assembly 580′.

As described above, FIG. 8 illustrates a first path flow position 686 in which fluid including the particulate 660 as the mixed solution 662 is directed along the first path 626A, 626 disposed between the particulate region 604 and the neck region 602 of the vial assembly 580′ absent a high concentrate of underlying and unsuspended particulate 660 in the vial assembly 580′. Thus, the first path 626A, 626 of FIG. 8 may be used when the bypass valve 606 is not triggered to divert the mixed solution 662 along the bypass path 624 of FIG. 7 to prevent clogging.

In embodiments, a method of operating the vial assembly system 680 may thus include receiving the needle 559 in the septum 592 of the vial assembly 580′ of the vial assembly system 680 such that the port 614 of the needle 559 is disposed in the neck region 602 of the vial assembly 580′. A pressure build-up in the vial assembly 580′ may be monitored to determine whether the pressure build-up exceeds a threshold. When the pressure build-up that is monitored exceeds the threshold, a fluid flow path may be alternated from the first path 626 to the bypass path 624, such as shown in FIG. 7. As described above, the bypass path 624 includes the first end 620 and the second end 622, the bypass valve 606 is connected to the first end 620 of the bypass path 624, the second end 622 of the bypass path 624 ends at the sidewall 612 of the neck region 602, and the first path 626 is directly disposed in the vial assembly 580′ between the particulate region 604 and the neck region 602.

In embodiments, a different number, size, and/or location of concentration regulators 664 may be used with the vial assembly 580′. Additionally or alternatively, a filter may be added positioned before the bypass valve 606. The bypass valve 606 may be a one way or pop-off valve or other suitable valve as described herein made of a variety of materials, and the concentration regulator 664 may be made of a variety of metals, plastics, hydrogels, and the like.

### III. Aspects Listing

Aspect 1. A vial assembly system comprises a vial assembly and a needle. The vial assembly comprises a septum, a neck region, and a particulate region for containing particulates comprising a bypass valve, the neck region comprising a top, a bottom, and a sidewall disposed therebetween. The needle comprises a port and a tip distal of the port, the tip configured to puncture the septum of the vial assembly disposed at the bottom of the neck region. The septum is configured to receive the needle such that the port is disposed in the neck region of the vial assembly. A bypass path includes a first end and a second end. The bypass valve is connected to the first end of the bypass path, and the second end of the bypass path ends at or within the sidewall of the neck region. The bypass path is configured to provide a fluid flow path to the needle alternate to a first path upon a build-up of pressure for fluid flow in the vial assembly, and the first path is directly disposed in the vial assembly between the particulate region and the neck region.

Aspect 2. The vial assembly system of Aspect 1, further comprising a concentration regulator disposed between the neck region and the particulate region and configured to restrict a concentration of particulates to send along the first path to the needle.

Aspect 3. The vial assembly system of Aspect 2, wherein the concentration regulator comprises a mesh component

Aspect 4. The vial assembly system of any of Aspect 1 to Aspect 3, wherein the bypass path is configured to provide the fluid flow path to the needle alternate to the first path upon the build-up of pressure for fluid flow in the vial assembly when a build-up of particulates occur on the concentration regulator.

Aspect 5. The vial assembly system of any of Aspect 1 to Aspect 4, wherein the neck region comprises a cylindrical shape.

Aspect 6. The vial assembly system of any of Aspect 1 to Aspect 4, wherein the neck region comprises a conical shape.

Aspect 7. The vial assembly system of any of Aspect 1 to Aspect 6, wherein the bypass valve disposed in the particulate region is further disposed in a deadspace region of the particulate region, the deadspace region defined as an area into which a plunger of the vial assembly is unable to distally plunge such that the plunger is unable to plunge into the deadspace region.

Aspect 8. The vial assembly system of any of Aspect 1 to Aspect 7, wherein the port of the needle is configured as an outlet to inject fluid into the vial assembly and as an inlet to deliver a mixed particulate solution from the vial assembly.

Aspect 9. The vial assembly system of any of Aspect 1 to Aspect 8, wherein the first path is directed along a longitudinal axis of the vial assembly, and the bypass path is radially disposed with respect to the longitudinal axis.

Aspect 10. The vial assembly system of any of Aspect 1 to Aspect 9, wherein the bypass path comprises a tubing disposed between the bypass valve and the sidewall of the neck region.

Aspect 11. The vial assembly system of any of Aspect 1 to Aspect 10, wherein the septum is configured to be disposed proximally adjacent to the neck region, and the particulate region is disposed distally adjacent to the neck region.

Aspect 12. A vial assembly system comprises a vial assembly and a needle. The vial assembly comprises a septum, a neck region, and a particulate region for containing particulates comprising a bypass valve, the neck region comprising a top, a bottom, and a sidewall disposed therebetween, and the needle comprises a port and a tip distal of the port, the tip configured to puncture the septum of the vial assembly disposed at the bottom of the neck region. The septum is configured to receive the needle such that the port is disposed in the neck region of the vial assembly. The bypass valve is connected to a bypass path including a first end and a second end. The bypass valve is connected to the first end of the bypass path, and the second end of the bypass path ends at or within the sidewall of the neck region. The bypass path is configured to provide a fluid flow path to the needle alternate to a first path upon a build-up of pressure for fluid flow in the vial assembly, and the first path is directly disposed in the vial assembly between the particulate region and the neck region. The bypass path comprises a tubing disposed between the bypass valve and the sidewall of the neck region, and the tubing of the bypass path is radially disposed with respect to a longitudinal axis of the vial assembly.

Aspect 13. The vial assembly system of Aspect 12, further comprising a concentration regulator disposed between the neck region and the particulate region and configured to restrict a concentration of particulates to send along the first path to the needle.

Aspect 14. The vial assembly system of Aspect 13, wherein the concentration regulator comprises a mesh component.

Aspect 15. The vial assembly system of any of Aspect 12 to Aspect 14, wherein the bypass path is configured to provide a fluid flow path to the needle alternate to the first path upon the build-up of pressure for fluid flow in the vial assembly when a build-up of particulates occur on the concentration regulator.

Aspect 16. The vial assembly system of any of Aspect 12 to Aspect 15, wherein the neck region comprises a cylindrical shape.

Aspect 17. The vial assembly system of any of Aspect 12 to Aspect 15, wherein the neck region comprises a conical shape.

Aspect 18. The vial assembly system of any of Aspect 12 to Aspect 17, wherein the bypass valve is disposed in the particulate region is further disposed in a deadspace region of the particulate region, the deadspace region defined as an area into which a plunger of the vial assembly is unable to distally plunge such that the plunger is unable to plunge into the deadspace region.

Aspect 19. The vial assembly system of any of Aspect 12 to Aspect 18, wherein the port of the needle is configured as an outlet to inject fluid into the vial assembly and as an inlet to deliver a mixed particulate solution from the vial assembly.

Aspect 20. A method of operating a vial assembly system comprises receiving a needle in a septum of a vial assembly of the vial assembly system such that a port of the needle is disposed in a neck region of the vial assembly. The vial assembly further comprises a particulate region comprising a bypass valve, the neck region comprising a top, a bottom, and a sidewall disposed therebetween, the needle further comprising a tip distal of the port, the tip configured to puncture the septum of the vial assembly disposed at the bottom of the neck region. The method further comprises monitoring a pressure build-up in the vial assembly to determine whether the pressure build-up exceeds a threshold, and, when the pressure build-up that is monitored exceeds the threshold, alternating a fluid flow path from a first path to a bypass path. The bypass path includes a first end and a second end, the bypass valve is connected to the first end of the bypass path, the second end of the bypass path ends at or within the sidewall of the neck region, and the first path is directly disposed in the vial assembly between the particulate region and the neck region.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

For the purposes of describing and defining the present disclosure it is noted that the term “substantially” is used herein to represent the inherent degree of uncertainty that may be attributed to any quantitative comparison, value, measurement, or other representation. The term “substantially” is used herein also to represent the degree by which a quantitative representation may vary from a stated reference without resulting in a change in the basic function of the subject matter at issue. As such, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something slightly less than exact.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A vial assembly system, comprising:
a vial assembly comprising a septum, a neck region, and a particulate region for containing particulates comprising a bypass valve, the neck region comprising a top, a bottom, and a sidewall disposed therebetween;
a needle comprising a port and a tip distal of the port, the tip configured to puncture the septum of the vial assembly disposed at the bottom of the neck region;
wherein the septum is configured to receive the needle such that the port is disposed in the neck region of the vial assembly; and
wherein a bypass path includes a first end and a second end, the bypass valve is connected to the first end of the bypass path, the second end of the bypass path ends at or within the sidewall of the neck region, the bypass path is configured to provide a fluid flow path to the needle alternate to a first path upon a build-up of pressure for fluid flow in the vial assembly, and the first path is directly disposed in the vial assembly between the particulate region and the neck region.

2. The vial assembly system of claim 1, further comprising a concentration regulator disposed between the neck region and the particulate region and configured to restrict a concentration of particulates to send along the first path to the needle.

3. The vial assembly system of claim 2, wherein the concentration regulator comprises a mesh component.

4. The vial assembly system of claim 2, wherein the bypass path is configured to provide the fluid flow path to the needle alternate to the first path upon the build-up of pressure for fluid flow in the vial assembly when a build-up of particulates occur on the concentration regulator.

5. The vial assembly system of claim 1, wherein the neck region comprises a cylindrical shape.

6. The vial assembly system of claim 1, wherein the neck region comprises a conical shape.

7. The vial assembly system of claim 1, wherein the bypass valve disposed in the particulate region is further disposed in a deadspace region of the particulate region, the deadspace region defined as an area into which a plunger of the vial assembly is unable to distally plunge such that the plunger is unable to plunge into the deadspace region.

8. The vial assembly system of claim 1, wherein the port of the needle is configured as an outlet to inject fluid into the vial assembly and as an inlet to deliver a mixed particulate solution from the vial assembly.

9. The vial assembly system of claim 1, wherein the first path is directed along a longitudinal axis of the vial assembly, and the bypass path is radially disposed with respect to the longitudinal axis.

10. The vial assembly system of claim 1, wherein the bypass path comprises a tubing disposed between the bypass valve and the sidewall of the neck region.

11. The vial assembly system of claim 1, wherein the septum is configured to be disposed proximally adjacent to the neck region, and the particulate region is disposed distally adjacent to the neck region.

12. A vial assembly system, comprising:
a vial assembly comprising a septum, a neck region, and a particulate region for containing particulates comprising a bypass valve, the neck region comprising a top, a bottom, and a sidewall disposed therebetween;
a needle comprising a port and a tip distal of the port, the tip configured to puncture the septum of the vial assembly disposed at the bottom of the neck region;
wherein the septum is configured to receive the needle such that the port is disposed in the neck region of the vial assembly; and
wherein the bypass valve is connected to a bypass path including a first end and a second end, the bypass valve connected to the first end of the bypass path, the second end of the bypass path ending at or within the sidewall of the neck region, the bypass path configured to provide a fluid flow path to the needle alternate to a first path upon a build-up of pressure for fluid flow in the vial assembly, the first path directly disposed in the vial assembly between the particulate region and the neck region, the bypass path comprises a tubing disposed between the bypass valve and the sidewall of the neck region, and the tubing of the bypass path is radially disposed with respect to a longitudinal axis of the vial assembly.

13. The vial assembly system of claim 12, further comprising a concentration regulator disposed between the neck region and the particulate region and configured to restrict a concentration of particulates to send along the first path to the needle.

14. The vial assembly system of claim 13, wherein the concentration regulator comprises a mesh component.

15. The vial assembly system of claim 13, wherein the bypass path is configured to provide a fluid flow path to the needle alternate to the first path upon the build-up of pressure for fluid flow in the vial assembly when a build-up of particulates occur on the concentration regulator.

16. The vial assembly system of claim 12, wherein the neck region comprises a cylindrical shape.

17. The vial assembly system of claim 12, wherein the neck region comprises a conical shape.

18. The vial assembly system of claim 12, wherein the bypass valve is disposed in the particulate region is further disposed in a deadspace region of the particulate region, the deadspace region defined as an area into which a plunger of the vial assembly is unable to distally plunge such that the plunger is unable to plunge into the deadspace region.

19. The vial assembly system of claim 12, wherein the port of the needle is configured as an outlet to inject fluid into the vial assembly and as an inlet to deliver a mixed particulate solution from the vial assembly.

20. A method of operating a vial assembly system, the method comprising:
receiving a needle in a septum of a vial assembly of the vial assembly system such that a port of the needle is disposed in a neck region of the vial assembly, the vial assembly further comprising a particulate region comprising a bypass valve, the neck region comprising a top, a bottom, and a sidewall disposed therebetween, the needle further comprising a tip distal of the port, the tip configured to puncture the septum of the vial assembly disposed at the bottom of the neck region;
monitoring a pressure build-up in the vial assembly to determine whether the pressure build-up exceeds a threshold; and
when the pressure build-up that is monitored exceeds the threshold, alternating a fluid flow path from a first path to a bypass path, wherein the bypass path includes a first end and a second end, the bypass valve is connected to the first end of the bypass path, the second end of the bypass path ends at or within the sidewall of the neck region, and the first path is directly disposed in the vial assembly between the particulate region and the neck region.
